(19) United States
(12) Patent Application Publication
(10) Pub. No.: US 2011/0022099 A1
(43) Pub. Date: Jan. 27, 2011

(54) Shape-Transforming Implant Device

(76) Inventor: Richard B. Ashman, New Orleans, LA (US)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 12/377,436

(22) PCT Filed: Aug. 16, 2007
(86) PCT No.: PCT/US07/76101

§ 371 Date: Oct. 12, 2010

Related U.S. Application Data
(60) Provisional application No. 60/838,772, filed on Aug. 18, 2006.

(51) Int. Cl. A61B 17/84

(52) U.S. Cl. 606/331

(57) ABSTRACT

A shape-transforming implant device for joining and holding in place a severed sternum. The device includes a bridge portion, two or more legs extending from the bridge portion, shoulder portions, and feet portions. The bridge portions have a thickness that is sized such that it can be cut with a pin cutter. The two or more legs extend from the bridge portion at a first angle when the bridge and the two or more legs are at a first temperature. The shoulder portions are defined by a first angle between the bridge portion and the two or more legs and define a transition from the bridge portion to the two or more legs. The feet portions are at an end of the two or more legs that is opposite from the shoulder portions. Each of the feet portions include an inwardly angled portion that extends toward the remaining feet portions.

### BACKGROUND OF THE INVENTION

(1) Field of the Invention

The present invention generally relates to surgical devices. In particular, the present invention is directed to methods and devices for joining and fusing a severed sternum.

(2) Description of the Related Art

The sternum is a flat elongated bone, which forms the middle portion of the anterior wall of the thorax. Its upper end supports the clavicles and its sides attach the cartilage of the first seven pairs of ribs. The sternum articulates on either side with the clavicle and upper seven costal cartilages. The bone material of the sternum consists of an outer shell of cortical bone covering the less dense cancellous bone.

While fractures of the sternum are not common, the sternum is cut or osteotomized, in a procedure called a stenotomy, to gain access to the organs of the chest, most commonly the heart. A partial sternotomy is a procedure in which a cut is made along the midline of the sternum and the two opposing sternal halves to be separated laterally. The opening thus created allows the surgeon direct visualization to be able to operate upon the heart and other thoracic organs or tissues. Following the sternotomy, the two severed sternal halves must be reapproximated, or rejoined.

When the sternum is closed, the sternal halves are brought together and held in place with stainless steel wire sutures. The wire is wrapped around the bone parts through the intercostal spaces. Surgeons have tried various suture-tying patterns over many years. Regardless of the method used, the complication rate of the sternal closure is significant. When closing the sternum with stainless steel wire, complication rates of 10% to 15% are typical. Overall rewiring is required in approximately 5% of patients. For obese patients, the rate increases to approximately 30% and for patients with severe lung disease, the rate of complication increases to 50%. Many patients complain of pain, or of a sense of motion, between the segments of the sternum.

Of importance to cardiac surgeons, and those who practice to medical art of surgically closing a sternum is the ability to quickly reopen the sternum if the need arises. Stainless steel wire sutures offer the ability of the surgeon to quickly and easily cut the wire if re-operation is necessary. Unfortunately, as mentioned above, there are disadvantages to using stainless steel wire.

### BRIEF SUMMARY OF THE INVENTION

One aspect of the present invention is a shape-transforming implant device for joining and holding in place a severed sternum. The device includes a bridge portion, two or more legs extending from the bridge portion, shoulder portions, and feet portions. The bridge portions have a thickness that is sized such that it can be cut with a pin cutter. The two or more legs extend from the bridge portion at a first angle when the bridge and the two or more legs are at a first temperature. The shoulder portions are defined by a first angle between the bridge portion and the two or more legs and define a transition from the bridge portion to the two or more legs. The feet portions are at an end of the two or more legs. The end is opposite from the shoulder portions. Each of the feet portions include an inwardly angled portion that extends toward the remaining feet portions. The feet portions are configured to stabilize the device once it has been placed around the sternum and its shape transformed.

### BRIEF DESCRIPTION OF THE DRAWINGS

For the purpose of illustrating the invention, the drawings show a form of the invention that is presently preferred. However, it should be understood that the present invention is not limited to the precise arrangements and instrumentalities shown in the drawings, wherein:

FIG. 1a is a front isometric view of a device in its opened shape according to embodiments of the disclosed subject matter;

FIG. 1b is a front isometric view of the device shown in FIG. 1a in its closed shape according to embodiments of the disclosed subject matter;

FIG. 1c is a front isometric view of the device shown in FIG. 1a in its closed shape around the lateral aspects of the sternum;

FIG. 2a is a front isometric view of a pair of the device shown in FIG. 1a spanning a patient's sternum;

FIG. 2b is a back isometric view of a pair of the device shown in FIG. 1a spanning a patient's sternum;

FIG. 3a is a front isometric view of a device in its opened shape according to embodiments of the disclosed subject matter;

FIG. 3b is a front isometric view of a device in its opened shape according to embodiments of the disclosed subject matter;

FIG. 3c a front isometric view of the devices shown in FIGS. 3a and 3b spanning a patient's sternum; and

FIG. 4 is a front isometric view of a device with a plurality of legs spanning a patient's sternum;

FIG. 5a is a front isometric view of a device in its opened shape according to embodiments of the disclosed subject matter;

FIG. 5b is a front isometric view of the device shown in FIG. 5a in its closed shape according to embodiments of the disclosed subject matter;

FIG. 5c is the device shown in FIG. 5a with legs that only penetrate the anterior cortex of the sternum;

FIG. 5d is the device shown in FIG. 5a with legs long enough to penetrate both cortexes of the sternum;

FIG. 6a is a front isometric view of a device in its opened shape according to embodiments of the disclosed subject matter;

FIG. 6b is a front isometric view of the device shown in FIG. 6a in its closed shape according to embodiments of the disclosed subject matter; and

FIG. 6c is the device shown in FIG. 6a with legs configured to clamp around the lateral aspects of the sternum and legs configured to penetrate one or both cortexes of the sternum.

### DETAILED DESCRIPTION

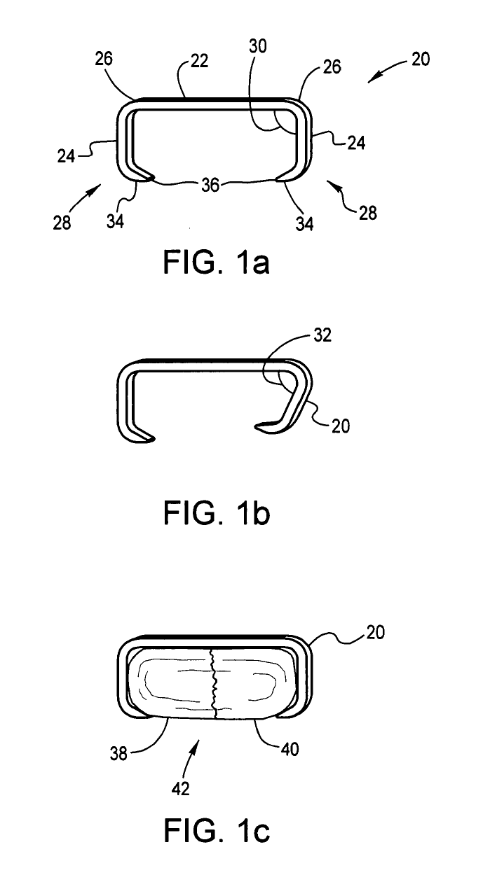

Referring now to the drawings in which like reference numerals indicate like parts, and in particular, to FIGS. 1-8, one aspect of the present invention is a shape-transforming implant device for joining and holding in place a severed sternum.

The shape-transforming implant device is typically formed from a shape memory material such those from a family of intermetallic materials named nitinol, an acronym for NIckel TItanium Naval Ordnance Laboratory. The properties of nitinol allow the material to exist in two stable phases. The lower temperature phase is referred to as the martensitic phase, due to its metallic structure being composed primarily of martensite. The higher temperature phase is referred to as the austenitic phase. Nitinol is an alloy composed primarily of nickel and titanium. By varying the percentage of nickel and titanium, different transition temperatures can be achieved. Transformation temperatures can be either below that of body temperature or slightly greater than body temperature. In some embodiments, the device is formed from a shape memory material having a transformation temperature below that of body temperature. In other embodiments, the device is formed from a shape memory material having a transformation temperature above that of body temperature

Referring now to FIGS. 1a-1c, in one embodiment, a device 20 includes a bridge portion 22, two or more legs 24 extending from the bridge portion via shoulder portions 26, and feet portions 28 at ends of the legs.

Bridge portion 22 generally has a thickness that is sized such that it can be cut with a standard surgical wire or pin cutter that is commonly used in orthopedic surgery. Two or more legs 24 extend from bridge portion 22 at a first angle 30 when the bridge and the two or more legs are at a first temperature. As best illustrated in FIG. 1b, two or more legs 24 extend from bridge portion 22 at a second angle 32 when the bridge and the two or more legs are at a second temperature that exceeds the transformation temperature.

Shoulder portions 26 are defined by first angle 30 between bridge portions 22 and two or more legs 24. Shoulder portions 26 define a transition from bridge portion 22 to two or more legs 24.

Feet portions 28 are formed at an end 34 of two or more legs 24 that is opposite from shoulder portions 26. Each of feet portions 28 includes an inwardly angled portion 36 that extends toward the remaining feet portions. Feet portions 28 are configured to stabilize device 20 once it has been placed around the sternum and its shape transformed.

Referring now to FIG. 1c, in some embodiments, device 20 includes two legs 24 that are configured to clamp around lateral aspects 38 and 40 of a severed sternum 42. Two legs 24 are configured to deflect inwardly (as reflected by arrows) at a second temperature above the transformation temperature, thereby compressing lateral aspects 38 and 40 of severed sternum 42.

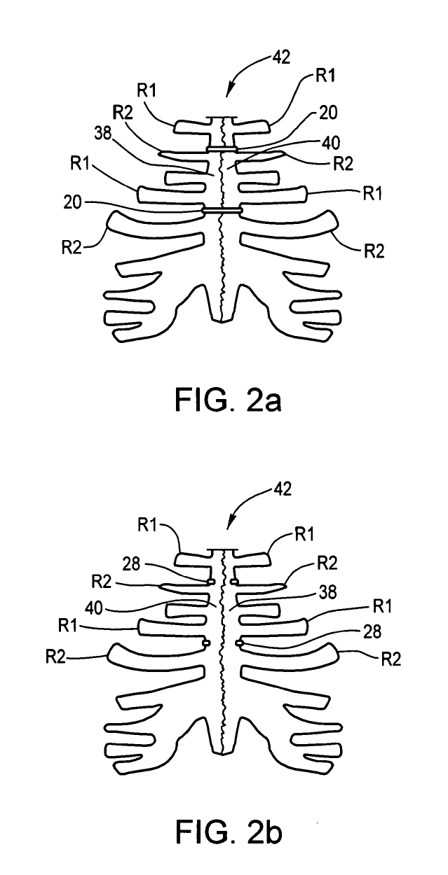

Referring now to FIGS. 2a and 2b, in use, legs 24 of devices 20 are positioned intercostally between two corresponding rib pairs R1 and R2. In FIG. 2a, bridge portions 22 of devices 20 span across the frontal surfaces (not numbered) of lateral aspects 38 and 40 of severed sternum 42. In FIG. 2b, feet portions 28 of devices 20, which are positioned intercostally between two corresponding rib pairs R1 and R2, wrap-around and onto posterior surfaces (not numbered) of lateral aspects 38 and 40 of severed sternum 42.

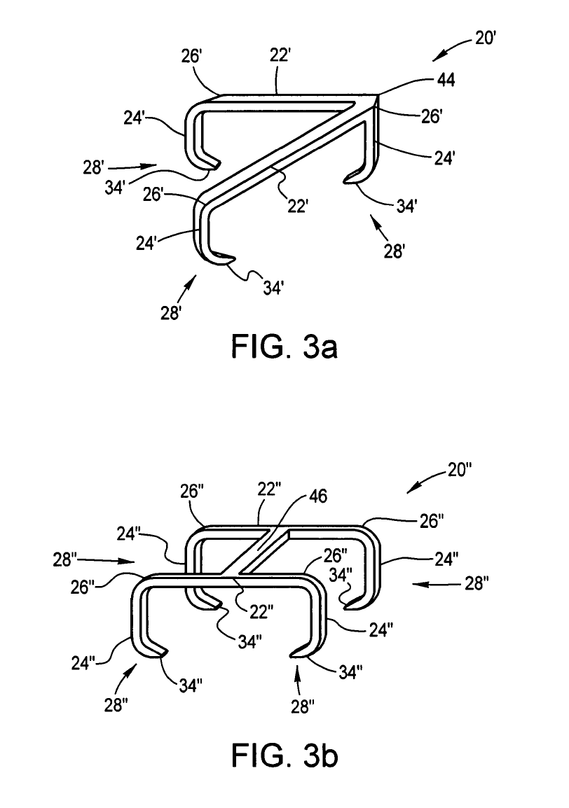

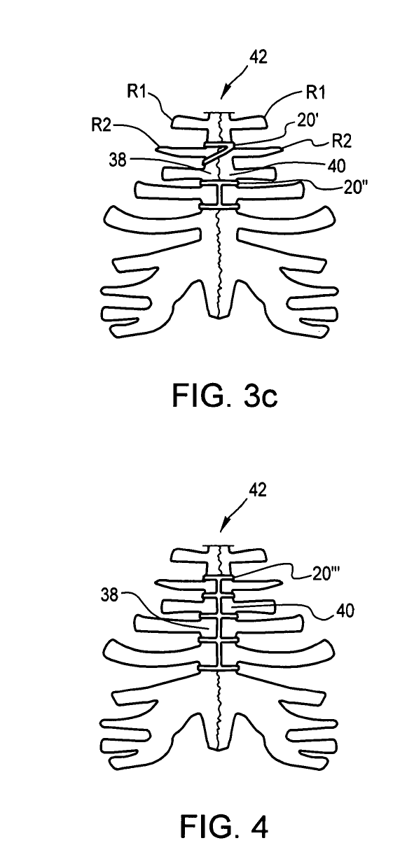

Referring now to FIGS. 3a-3c and 4, alternative embodiments closely related to the embodiments illustrated in FIG. 1c can include additional legs. Additional legs provide additional longitudinal stability to the reapproximated sternum. As shown in FIG. 3a, a device 20′ can include three legs 24′ and two bridge portions 22′ that are joined at a variable joining point 44 depending on the geometry of the application. As with device 20, legs 24′ include feet portions 28′ formed at ends 34′ that are opposite from shoulder portions 26′.

Referring now to FIG. 3b, a device 20″ can include three legs 24″ and two bridge portions 22″ that are joined via a variable crossbar member 46 depending on the geometry of the application. As with devices 20 and 20′, legs 24″ include feet portions 28″ formed at ends 34″ that are opposite from shoulder portions 26″.

Referring now to FIG. 3c, in use devices 20′ and 20″ are positioned along severed sternum 42. As illustrated in FIG. 4, in other embodiments, a device 20′″ can include as many legs 24′″ as intercostal spaces (not numbered) to close severed sternum 42.

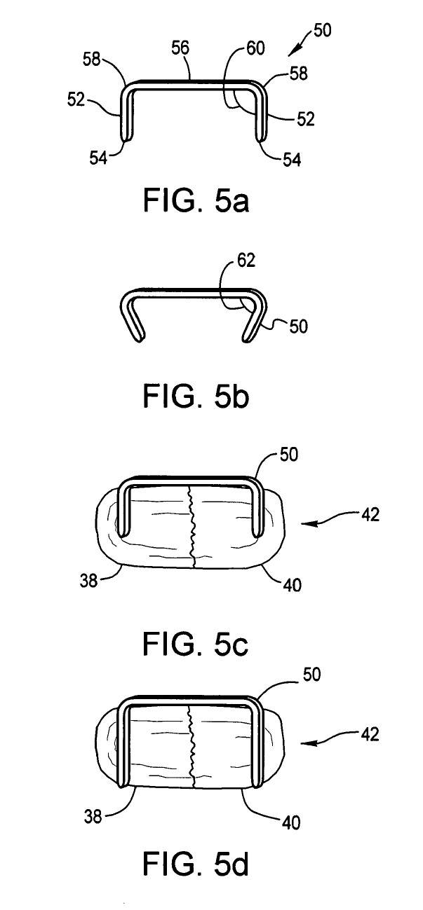

Referring now to FIGS. 5a-5d, in some embodiments, a device 50 includes two legs 52 that are configured to be inserted into severed sternum 42. Two legs 52 include ends 54 that do not have inwardly angled feet. Rather, ends 54 are straight and can end in a point (not shown) to facilitate insertion into severed sternum 42. A bridge portion 56 connects two legs 52. Two legs 52 extend from bridge portion 56 via shoulder portions 58 at a first angle 60 when the bridge portion and the two legs are at a first temperature. As best illustrated in FIG. 5b, two legs 52 extend from bridge portion 56 via shoulder portions 58 at a second angle 62 when the bridge portion and the two legs are at a second temperature that exceeds the transformation temperature.

As shown in FIG. 5c, in some embodiments, two legs 52 are configured to only extend through an anterior cortex 54 of lateral aspects 38 and 40 of severed sternum 42. As shown in FIG. 5d, in other embodiments, two legs 52 are configured to extend through both anterior cortex 54 and a posterior cortex 56 of lateral aspects 38 and 40 of severed sternum 42. Again, two legs 52 are configured to deflect inwardly (as reflected by arrows) at a second temperature above the transformation temperature, thereby compressing lateral aspects 38 and 40 of severed sternum 42. Although not illustrated, in some embodiments, device 50 can include three or more legs that are configured to be inserted into severed sternum 42.

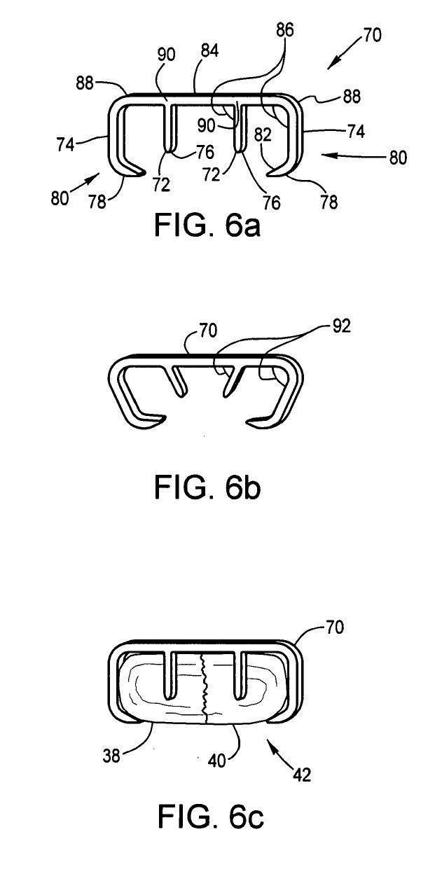

Referring now to FIGS. 6a-6c, in some embodiments, a device 70 includes two legs 72 that are configured to be inserted into severed sternum 42 and two legs 74 that are configured to clamp around lateral extents 38 and 40 of the severed sternum. Two legs 72 include ends 76 that do not have inwardly angled feet. Rather, ends 76 are straight and can end in a point (not shown) to facilitate insertion into severed sternum 42. In contrast, ends 78 of two legs 74 include feet portions 80 having inwardly angled portions 82 that extend toward the opposite feet portions.

A bridge portion 84 connects all of legs 72 and 74 to one another. Two legs 72 and 74 extend from bridge portion 84 at a first angle 86 when the bridge portion and the two legs are at a first temperature. Two legs 74 extend from bridge portion 84 via shoulder portions 88. Two legs 72 extend from bridge portion 84 at predetermined points 90 along the bridge portion that are interior to shoulder portions 88. The geometry of severed sternum 42 governs the location of predetermined points 90. As best illustrated in FIG. 6b, two legs 72 and 74 extend from bridge portion 84 at a second angle 92 when the bridge portion and the two legs are at a second temperature that exceeds the transformation temperature, thereby compressing lateral aspects 38 and 40 of severed sternum 42.

As shown in FIG. 6c, in some embodiments, two legs 72 are configured to only extend through anterior cortex 54 of lateral aspects 38 and 40 of severed sternum 42. Although not illustrated, in other embodiments, two legs 72 can be configured to extend through both anterior cortex 54 and posterior cortex 56 of lateral aspects 38 and 40 of severed sternum 42. Again, two legs 72 are configured to deflect inwardly (as reflected by arrows) at a second temperature above the transformation temperature, thereby compressing lateral aspects 38 and 40 of severed sternum 42. Although not illustrated, in some embodiments, device 70 can include three or more legs that are configured to be inserted into severed sternum 42.

In use, if a device is formed from nitinol with a transition temperature below body temperature, the procedure to implant the device would require the device to be cooled below its transition temperature. The legs would then be spread apart with the shoulders spread opened as in FIG. 1a. After the device has been placed around the sternum, the warmth of the body warms the device above its transition temperature. Above the transition temperature, the shape of the device attempts to revert back to the high temperature shape as generally shown in FIG. 1b. The shoulders are shown transformed into the device's closed shape. The final shape of the device is determined both by the austenitic shape formed into the device, and also the limits on closure imposed by the geometry of the sternum. The device imposes a compressive force across the sternotomy once the device has been transformed and closed.

In use, if the device is comprised of nitinol with a transition temperature slightly above body temperature, the device is provided to a surgeon in its low temperature, i.e., martensitic state. In this state, the legs of the device would be spread apart as depicted in FIG. 1a. After the device has been placed around the sternum, the material would be warmed using an external heater as the one disclosed by Flot in U.S. Pat. No. 6,268,589.

Once the nitinol material has warmed above its transition temperature, the device's shape will attempt to revert back to the high temperature shape as generally shown in FIG. 1b. The clamp material would then lower to that of body temperature. Shape memory materials such as Nitinol exhibit a temperature transformation hysterisis. This causes the temperature of transformation when moving from the low temperature phase to the high temperature phase to be greater than the transformation temperature when moving form the higher temperature phase to the low temperature phase. The body's temperature would thus keep the material from transforming back to the lower temperature phase since body temperature would be greater than the transformation temperature from the high temperature phase to the lower temperature phase. The final shape of the device is determined both by the austenitic shape formed into the device, and also the limits on closure imposed by the geometry of the sternum.

Although the invention has been described and illustrated with respect to exemplary embodiments thereof, it should be understood by those skilled in the art that the foregoing and various other changes, omissions and additions may be made therein and thereto, without parting from the spirit and scope of the present invention. Accordingly, other embodiments are within the scope of the following claims.

What is claimed is:

1. A shape-transforming implant device for joining and holding in place a severed sternum, said device comprising:
a bridge portion having a thickness that is sized such that it can be cut with a pin cutter;
two or more legs extending from said bridge portion at a first angle when said bridge and said two or more legs are at a first temperature;
shoulder portions defined by said first angle between said bridge portion and said two or more legs, said shoulder portions defining a transition from said bridge portion to said two or more legs; and
feet portions at an end of said two or more legs that is opposite from said shoulder portions, each of said feet portions including an inwardly-angled portion that extends toward others of said feet portions, wherein said feet portions are configured to stabilize the device once it has been placed around the sternum and its shape transformed.

2. A device according to claim 1, wherein said device is formed from a shape memory material.

3. A device according to claim 2, wherein said shape memory material includes a transformation temperature below that of body temperature.

4. A device according to claim 2, wherein said shape memory material includes a transformation temperature above that of body temperature.

5. A device according to claim 2, wherein said two or more legs are configured to deflect inwardly at a second temperature above said transformation temperature, thereby compressing the severed sternum.

6. A device according to claim 2, wherein said device includes two legs that are configured to clamp around lateral aspects of the severed sternum.

7. A device according to claim 6, wherein said two legs are configured to deflect inwardly at a second temperature above said transformation temperature, thereby compressing the severed sternum.

8. A device according to claim 6, wherein said second temperature is above said first temperature and a second angle less than said first angle is formed at said second temperature.

9. A device according to claim 2, wherein said device includes two legs that are configured to be inserted into the body of the severed sternum.

10. A device according to claim 9, wherein said two legs are configured to deflect inwardly at a second temperature above said transformation temperature, thereby compressing the severed sternum.

11. A device according to claim 10, wherein said second temperature is above said first temperature and a second angle less than said first angle is formed at said second temperature.

12. A device according to claim 9, wherein said two legs are configured to only extend through the anterior cortex of the severed sternum.

13. A device according to claim 9, wherein said two legs are configured to extend through both the anterior and posterior cortexes of the severed sternum.

14. A device according to claim 2, wherein said device includes three legs that are configured to be inserted into the body of the severed sternum.

15. A device according to claim 14, wherein said three legs are configured to deflect inwardly at a second temperature above said transformation temperature, thereby compressing the severed sternum.

16. A device according to claim 15, wherein said second temperature is above said first temperature and a second angle less than said first angle is formed at said second temperature.

17. A device according to claim 1, wherein said device includes four legs, said four legs including two legs that are configured to be inserted into the body of the severed sternum and two legs that are configured to clamp around lateral extents of the severed sternum.

18. A device according to claim 14, wherein said four legs are configured to deflect inwardly at a second temperature above said transformation temperature, thereby compressing the severed sternum.

19. A device according to claim 18, wherein said second temperature is above said first temperature and a second angle less than said first angle is formed at said second temperature.

20. A device according to claim 17, wherein said two legs are configured to only extend through the anterior cortex of the severed sternum.

21. A device according to claim 17, wherein said two legs are configured to extend through both the anterior and posterior cortexes of the severed sternum.
